(19) United States
(12) Patent Application Publication
(10) Pub. No.: US 2016/0299083 A1
(43) Pub. Date: Oct. 13, 2016

(54) GAS SENSOR

(71) Applicants: UNIVERSITE DE MONS, Mons (BE); MATERIA NOVA, Mons (BE)

(72) Inventors: Antonio Bueno Martinez, Mons (BE); Christophe Caucheteur, Mons (BE); Marc Debliquy, Mons (BE); Driss Lahem, Mons (BE); Patrice Megret, Mons (BE); Marie-Georges Olivier, Mons (BE)

(21) Appl. No.: 15/101,164

(22) PCT Filed: Dec. 1, 2014
(86) PCT No.: PCT/EP2014/076133

§ 371 Date: Jun. 2, 2016

(30) Foreign Application Priority Data
Feb. 12, 2013 (GB) 1321245.1

(51) Int. Cl. G01N 21/78; G01N 21/77; G01N 33/00

(52) U.S. Cl. CPC G01N 21/783; G01N 33/0037; G01N 33/0018; G01N 21/7703; G01N 2021/7709; G01N 2021/773; G01N 2021/7773; G01N 2021/758

(57) ABSTRACT

The present invention relates to gas sensors, in particular, to an optical fibre sensor for measuring the presence and/or quantity of one of more gasses, the gas sensor comprising an optical fibre, and a gas sensitive detection material at a portion of the surface of the optical fibre, said gas sensitive detection material comprising a gas sensitive reactant and a porous matrix, wherein the gas sensitive detection material undergoes a reversible change of reflectance and/or absorbance at a detection wavelength when subjected to a gas to be detected.

The present invention relates to gas sensors, in particular to an optical fibre sensor for measuring the presence and/or quantity of one of more gasses, notably in ambient air.

### BACKGROUND AND PRIOR ART

There exist numerous point sensors exploiting various technologies to detect gases, for example, electrochemical, infrared, semiconductor, pellistors and optical. There is nevertheless a need for improved gas detectors.

### SUMMARY OF THE INVENTION

According to one of its aspects, the present invention provides a gas sensor as defined in claim 1. Additional aspects are defined in other independent claims. The dependent claims define preferred or alternative embodiments.

The gas sensor according to the present invention comprises an optical fibre having a gas sensitive detection material at a portion of the external surface of the optical fibre. When exposed to a gas to be detected, the gas sensitive detection material, comprising a porous matrix and a gas sensitive reactant, undergoes a reversible change of absorbance and/or reflectance and/or refractive index at a detection wavelength.

The use of a sensor based on optical fibres provides various advantages. Historically, optical fibres were developed for long distance transmission of data and a whole technology was then developed to produce sources, detectors, spectrum analysers etc. in the telecom wavelength range which corresponds to the minimum of losses of silica fibres. The optical fibre based sensor may provide one or more of: immunity to interferences, possibilities of interrogation at numerous points on the same fibre, low weight and small volume, flexibility, stability, high temperature tolerance, durability, safety.

The optical fibre is preferably a silica fibre. This provides low attenuation, particularly at the preferred wavelengths referred to herein, is based on mature technology, can be used with common data processing equipment and is suitable for long distance transmission of data notable in the range of wavelengths 1300 nm-1700 nm, a range which corresponds to low signal losses for silica optical fibres. The silica may be a doped silica. Alternatively, the optical fibre may be a glass fibre or a polymer optical fibre, for example a PMMA (Poly(methyl methacrylate)) optical fibre.

The optical fibre is preferably a mono-mode optical fibre, also referred to as single-mode optical fibre. This facilitates retention of fidelity over long distances and allows use of a spectra having a structure which is fairly easy to interpret using standard equipment. Alternatively, the optical fibre may be a multi-mode optical fibre. The optical fibre may be a micro-structured optical fibre, notably photonic crystal fibre, a multicore optical fibre or a hollow core optical fibre.

In a preferred embodiment, the optical fibre is a single-mode silica optical fibre.

Preferably the optical fibre comprises an optical core and a cladding, both of which may be of silica. The core and/or the cladding may each be homogeneous.

The gas sensitive detection material may be provided in the form of a layer; it preferably has a significant change in reflectance and/or absorbance and/or refractive index in the range of wavelengths 1300 nm-1700 nm. This makes it particularly suitable for use with optical fibres, notably silica type optical fibres. The detection wavelength may be in the range from 300 nm to 1700 nm, preferably from 1100 nm to 1600 nm, more preferably from 1380 nm to 1550 nm.

The length of the optical fibre may be at least about 50 m, at least about 100 m, at least about 500 m or at least about 1 km.

The gas sensitive detection material may be arranged at a tip of the optical fibre and/or at at least one portion of the external surface of the optical fibre along the fibre's length. Notably, the gas sensitive detection material may be arranged at an external peripheral surface of the optical fibre at a position where the fibre cladding is not recessed. A plurality of spaced gas sensitive detection materials may be arranged along the length of the fibre. Such materials may be spaced by a distance of at least 5 m, at least 10 m, at least 20 m at least 50 m at least 100 m, at least 200 m or at least 500 m.

In a preferred embodiment, the gas sensitive detection material may be arranged on the external surface of the optical fibre at a position along the length of the optical fibre over an optical grating, notably a Fibre Bragg Grating (FBG), Long Period Fibre Grating (LPFG) or Tilted Fibre Bragg Grating. Preferably, the optical grating is a Tilted Fibre Bragg Grating; this may be used to intrinsically provide temperature-insensitive operation. The optical grating may be arranged within the core and/or within the cladding of the optical fibre.

The optical fibre may further comprise structures than can couple light from the fibre core to the cladding, for example etched optical fibre, D-shaped optical fibre, tapers or hybrid interferometric structures made, for example, by splicing optical fibres of different diameters. These structures may couple modes and/or evanescent waves to the surroundings.

When a plurality of spaced gas sensitive detection materials are provided, each may have its own associated optical grating and be arranged at spaced positions along the length of the optical fibre. This may be used for quasi-distributed sensing (as opposed to a single point measurement). For example, at least 5, 10 or 20 spaced gas sensitive detection materials may be provided along the length of the fibre. One or more temperature reference indicators may be provided as part of the sensor, for example, to enable an indication of and/or compensation for temperature, notably provided by gratings, preferably of the same type associated with the gas sensitive detection material(s).

The refractive index of the gas sensitive detection material may be in the range 1.3 to 1.6, preferably in the range 1.4 to 1.5. Preferably, the difference between the refractive index of the gas sensitive detection material and the refractive index of the optical fibre at an interface between the gas sensitive detection material and the optical fibre is less than 15%, preferably less than 10%, more preferably less than 5%, notably at the detection wavelength(s). This reduces undesired reflection at the detection wavelength(s) at this interface.

The gas sensitive detection material may have a thickness which is at least 50 nm, preferably at least 500 nm and/or no more than 15 μm, preferably no more than 5 μm. Particularly in the case of an inorganic and/or sol gel matrix, the thickness is preferably no more than 2 μm. If the gas sensitive detection material is too thick it may have a tendency to have or develop cracks or the diffusion may be too long causing an increase of the response time. If it is too thin, notably with respect to the detection wavelength(s), the amount of the reversible change of absorbance and/or reflectance may be too weak to be easily detect by signal processing equipment.

The gas sensitive detection material and/or gas sensitive reactant may have a molar absorptivity of at least 5×10⁵ m⁻¹·mol⁻¹·l ⁻¹, preferably of at least 1×10⁶ m⁻¹·mol⁻¹·l⁻¹ at a detection wavelength of 1550 nm. The gas sensitive reactant may have a molar absorptivity of at least 1×10⁶ m⁻¹·mol⁻¹·⁻¹ preferably 1×10⁷ m⁻¹·mol⁻¹·l⁻¹ at a detection wavelength of 650 nm.

The gas sensitive detection material may have a length which is at least 2 , at least 5 mm, at least 1 cm, at least 5 cm or at least 10 cm and/or no more than 50 cm, no more than 30 cm or no more than 20 cm. The gas sensitive detection material preferably extends around the entire circumference of the optical fibre.

The use of a porous matrix as part of the gas sensitive detection material facilitates diffusion of the gas to be detected into the body of the gas sensitive detection material. This allows the gas to be detected to easily and quickly reach and interact with the gas sensitive reactant in the gas sensitive detection material. This improves the response time of the sensor.

The porous matrix may be an inorganic matrix, notably a matrix of a mineral material, preferably comprising or consisting essentially of silica. It may be a sol-gel matrix. The porous matrix may be an organic matrix, notably a polymer matrix. It may be a hybrid inorganic/organic matrix. Preferably, the porous matrix is a silica matrix.

The porous matrix, notably before impregnation with the gas sensitive reactant, may have a porosity which is at least 25%, preferably at least 30% and/or which is no more than 70%, preferably no more than 60%, more preferably no more than 50%. The porosity represents the percentage space of pores in the total volume. If the porosity of the porous matrix before impregnation is too low then the matrix will only be able to contain a small quantity of the quantity of gas sensitive reactant; the change in the gas sensitive detection material will then be difficult to detect with signal processing equipment. If the porosity of the porous matrix before impregnation is too high, the mechanical properties of the porous matrix may be low and the structure of the matrix may collapse when loaded with a desired amount of gas sensitive reactant.

The gas sensitive detection material may have a porosity which is at least 15%, preferably at least 20% and/or no more than 60%, preferably no more than 40%.

The pores of the porous matrix may have an average diameter which is at least 4 nm, preferably at least 10 nm or at least 20 nm and/or no more than 100 nm, preferably no more than 80 nm.

Preferably, the pores of the porous matrix have a diameter that is at least 10 times smaller than the detection wavelength. This provides good homogeneity for detection of the change in the gas sensitive detection material and decreases scattering at the detection wavelength.

The gas sensitive reactant may comprise a lanthanide bisphtalocyanine, for example lutetium bisphthalocyanine (LuPc₂). This provides a reactant which is reversible, notably at ambient temperature. In addition, it provides a reactant that has a suitable change at the preferred detection wavelength(s). Preferably, the gas sensitive reactant is a chemical compound.

Preferably the gas sensitive reactant is insoluble in water and/or non-volatile and/or stable at operating temperatures of the sensor, for example from about −30° C. to about 45° C.; it is preferably non-soluble in common solvents, for example ethanol and/or not sensitive to humidity, notably relative humidity in the range 5-95%. Preferably, the gas sensitive reactant is non-responsive and/or non-reactive to oxygen O₂; this is particularly useful when the gas sensor is to be used for a gas to be detected in an oxygen containing gaseous atmosphere, notably air.

The gas sensitive reactant is preferably present in the form of a solid dispersed within the porous matrix, notably in the form of crystals. The diameter of the crystals, notably with respect to at least 90% of the crystals and preferably for the average diameter, may be less than 50 nm, preferably less than 30 nm, more preferably less than 10 nm. This provides a rapid response time for the gas sensitive detection material. The choice of the pore sizes referred to above facilitate obtaining the aforementioned crystal sizes.

The reversible change which the gas sensitive detection material undergoes is preferably a chemical reaction that proceeds in either direction by variation of the quantity of the gas to be detected to which it is exposed. Preferably, the reaction is reversible at the operating temperature of the sensor, notably at a temperature of from −30° C. to 45° C.

The gas sensitive reactant may be a neutral molecule, for example LuPc₂ which has an optical spectrum different to the optical spectrum of the oxidised form of the molecule, LuPc₂⁺ notably at the preferred detection wavelength(s). When the molecule is exposed to a gas, for example NO₂, the oxidation may be partial and equilibrated and the complex LuPc₂⁺/NO₂⁻ is formed. Without the gas, this complex reverts back to the initial composition, in this case, LuPc₂ and NO₂. For LuPc₂, in the visible spectrum, the neutral molecule is green, the oxidised form LuPc₂³⁰ is red and the reduced form LuPc₂⁻ is blue. The gas sensitive reactant may have at least three oxidation states, notably at least three stable oxidation states. The reaction may be reversed without other external influence at ambient atmosphere conditions. The speed of the reaction, notably reverting to the condition in the absence of the gas to be detected, may be increased by one or more external factors, for example by exposing the gas sensitive detection material to UV radiation, notably having a wavelength of less than about 400 nm, preferably of less than about 380 nm and/or greater than 10 nm, preferably greater than 100 nm. The gas sensitive detection material may be exposed to UV radiation by means of radiation which is introduced in to the optical fibre, for example periodically or when desired, and which may be directed to the gas sensitive detection material, for example by a grating. For example, UV radiation may be provided via the optical fibre by exploiting higher order modes of a grating, for example the harmonic at smaller wavelengths in the UV range with the fundamental harmonic being in the IR range. UV radiation may be provided from an external UV radiation source, for example a UV lamp directed towards an external surface of gas sensitive detection material. The UV radiation may provide energy to facilitate or accelerate reducing an oxidised form of the gas sensitive reactant.

Preferably, the reversibility of the sensor is such that the difference between the absorbance and/or reflectance and/or refractive index of the gas sensitive reactant between:

a) a condition prior to being exposed to the gas to be detected; and

b) a condition in which it has been exposed to the gas to be detected and is subsequently exposed to an atmosphere that does not include the gas to be detected;

is less than 20%, preferably less than 10%, more preferably less than 5%, notably at the detection wavelength(s) and notably after a period of less than 8 hours, preferably a period of less than 4 hours, less than 2 hours, less than 1 hour or less than 30 minutes, with or without external application of energy from an external source, preferably at ambient atmospheric conditions and notably at a 20° C. and 1 atmosphere in ambient or test air.

When the gas sensitive detection material is kept in conditions in which the gas to be detected is not present so as to become stable and is subsequently subjected to at least 10 ppm of a gas to be detected, the change of reflectance and/or absorbance and/or refractive index may be 10% in less than 10 minutes, preferably in less than 5 minutes, more preferably in less than 2 minutes.

The gas to be detected may comprise an oxidising gas, notably an oxidising gas selected from the group consisting of: a nitrogen oxide, notably NO₂, O₃ and mixtures thereof. Such detection may be useful for monitoring atmospheric pollution in air.

The gas to be detected may comprise a reducing gas, notably a reducing gas selected from the group consisting of: CO, NH₃, formaldehyde and mixtures thereof.

The sensor may detect concentration of at least 1 ppb and/or at least 5 ppb and/or at least 20 ppb and/or at least 100 ppb and/or at least 1 ppm and/or at least 10 ppm of the gas to be detected; it may detect a concentration of gas in the range 1-10 ppm.

Advantageously, the gas sensor according to the present invention may detect variation in absorbance or reflectance of at least 0.01 or 0.1 dB. The variation in optical absorbance or reflectance of the gas sensitive detection material at the detection wavelength between a first condition in which the sensor is detecting the gas to be detected and a second condition in which no gas to be detected is present may be at least 0.02, preferably at least 0.04 and more preferably at least 0.06.

The gas sensor of the present invention may be used for qualitative and/or quantitative measurements. It may detect the absolute amount of gas to be detected in the gaseous atmosphere and/or detect the relative amount or change in the amount of gas to be detected.

The gas sensor may comprises a gas filter, which may comprise activated carbon, arranged between the gas detection material and the gaseous atmosphere to filter one or more gasses to be detected and/or to reduce the concentration of the gas to be detected by the gas sensitive detection material.

A mechanical packaging may surround and/or block and/or be applied on or around the gas sensitive detection material, for example a metallic grid or a sintering, notably a ceramic sintering. The packaging may comprise a filter. For example the packaging may comprise a sintered material having a functioned surface provided by a filtering material or a metallic grid retaining a filter.

The gas sensor may be manufactured by:
Depositing the porous matrix at a portion of the external surface of an optical fibre; and
Subsequently impregnating the at least porous matrix with at least a gas reversible reactant.

This allows good control of distribution of the gas reversible reactant within the gas sensitive detection material.

The gas sensor may be used with a system comprising signal processing equipment which may transmit and/or detect and/or receive and/or analyse a signal at the detection wavelength(s). The signal processing equipment may comprise a light source and/or receiver or detector, for example an ASE (Amplified Spontaneous Emission) and/or a signal analyser for example an OSA (Optical Spectra Analyser). The light source may comprise a white light source, for example halogen lamp, laser diode, super luminescent laser diode, ASE source or wavelength tuneable laser. The detector may include one or more photodiode(s), power meter(s), optical spectrum analyser, and/or optical time domain reflectometer(s).

The transmitted and/or detected signal may be non-polarised or polarised. When polarised it is preferably polarised in P mode (which generally provides a better sensitivity than S mode) but it may be in S mode.

Preferably, the sensor is substantially insensitive to humidity. It is preferably humidity neutral, that is to say that the difference between the absorbance and/or reflectance of the sensor, between:

a) a condition wherein the humidity is 20% at a temperature of 20° C. and at a pressure of 1 atmosphere; and

b) a condition wherein the humidity is 80% at a temperature of 20° C. and at a pressure of 1 atmosphere; and

in which the gas sensor has been exposed to the same quantity of gas to be detected, notably 1ppm in air and/or 0ppm in air is less than 20%, preferably less than 10%, more preferably less than 5%, notably at the detection wavelength(s) and notably after a period of at least 8 hours, preferably a period of at least 4 hours, at least 2 hours, at least 1 hour or at least 30 minutes, with or without external application of energy from an external source.

The sensor is preferably used in ambient air, notably to measure or monitor air pollutant gasses. It may be used, for example, in road tunnels, car parks, storage halls, floor voids, cable ducts or sewers. It may be used for gas leak detection or for detecting or monitoring in a large open space. Where a single fibre having a plurality of spaced gas sensitive detection materials is used for air pollution and/or gas detection this provides an easily installed and cost efficient system for large areas.

### DESCRIPTION OF THE FIGURES

FIG. 1 is a schematic cross-section (not to scale) showing a gas sensor

FIG. 2 is a schematic cross-section (not to scale) showing an alternative gas sensor

FIG. 3 shows a TEM (Transmission Electron Microscopy) image of a porous matrix

FIGS. 4, 5, 6 and 7 are graphs showing the response of a gas sensor

FIG. 8 is a graph showing response of a material not in accordance with the invention

### DESCRIPTION OF PREFERRED EMBODIMENTS

### EXAMPLE 1

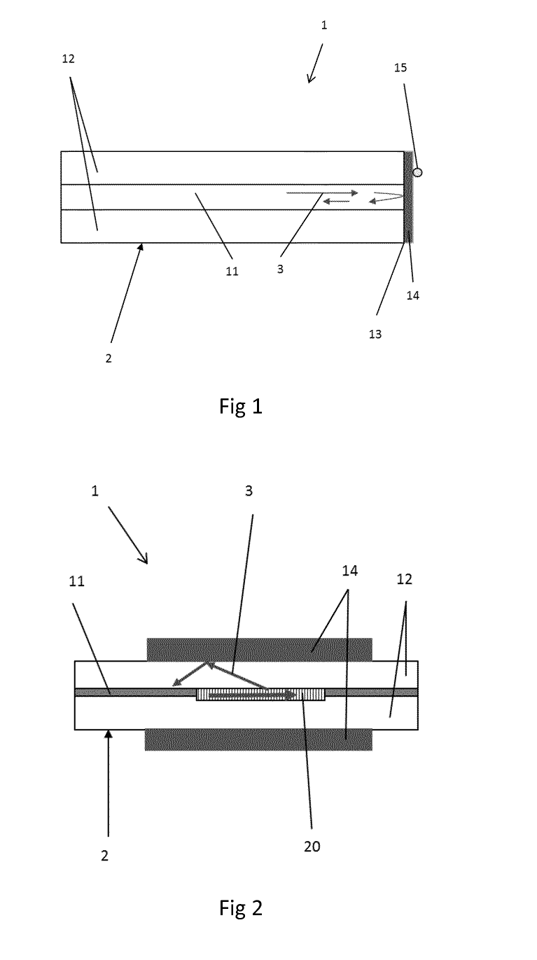

The optical fibre (2) shown in FIG. 1 comprises a cladding (12) and a core (11) and is a standard mono-mode fibre manufactured by Dow Corning. The core has a refractive index of 1.45 at a wavelength of 500 nm.

A gas sensitive detection material having a thickness of about 1 μm (14) is arranged on the surface of on a tip (13) of the optical fibre (2). A broad band ASE source (not shown) is connected at the other end of the fibre and transmits an incident wavelength spectrum (3) varying from 1200 nm to 1800 nm. The reflected spectrum is followed by means of an OSA (Optical Spectra Analyser). The resolution of the spectrum which is shown in FIG. 4 is 1 pico meter (pm). The detection wavelength is 1540 nm.

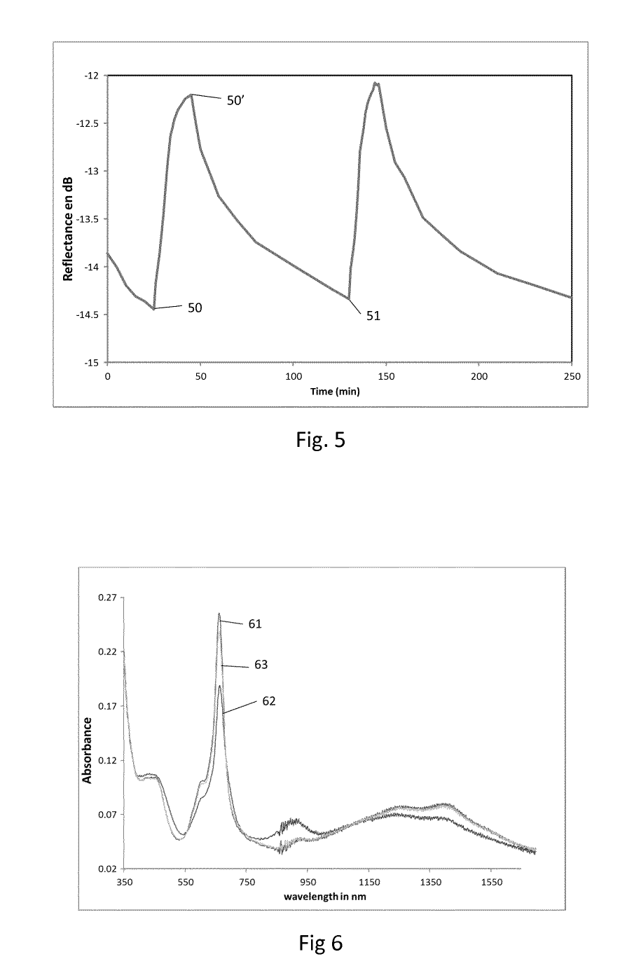

The sensor is held in a gas stream consisting of test air in a gas chamber containing controlled test air. The test air consists of about 79% nitrogen N₂ and about 21% oxygen O₂. For testing, the gaseous atmosphere is maintained at a temperature of 20° C., a pressure of about 1 atmosphere and a relative humidity of less than 5%. A concentration of 3 ppm of NO₂ is subsequently introduced into the stream of test air directed towards the sensor inside the gas chamber. The reflected light spectrum is analysed and provides an indication of the NO₂gas concentration. The results of reflectance are shown in FIG. 4 and FIG. 5.

### EXAMPLE 2

In the example shown in FIG. 2, the gas sensitive detection material (14) is arranged on the external surface of the optical fibre at a position along the length of the optical fibre over a Tilted Fibre Bragg Gratings (20).

One or more additional gas sensitive detection materials (not shown) each having its own associated Tilted Fibre Bragg Gratings may be arranged at spaced positions along the length of the optical fibre.

The reflected spectrum from a broad band ASE source which transmits a wavelength spectrum (3) varying from 1200 nm to 1800 nm through the optical fibre is followed by means of an OSA (Optical Spectra Analyser).

In each of the examples, the gas sensitive detection material comprises a porous matrix which consists of a porous silica deposited by sol-gel and having an average pore diameter of 50 nm which is impregnated with lutetium bisphthalocyanine (LuPc₂). The LuPc₂ fills about 33% of the pore volume. The gas sensitive detection material in these examples has an optical absorbance of about 0.06 at 1550 nm which is easily measured; the variations are of the order of 0.02 to 0.06. The LuPc₂ has a molar absorptivity of about 1.2×10⁶ m⁻¹·mol⁻¹·l⁻¹ at 1550 nm and about 3.0×10⁷ m⁻¹·mol⁻¹·l⁻¹ at 650 nm.

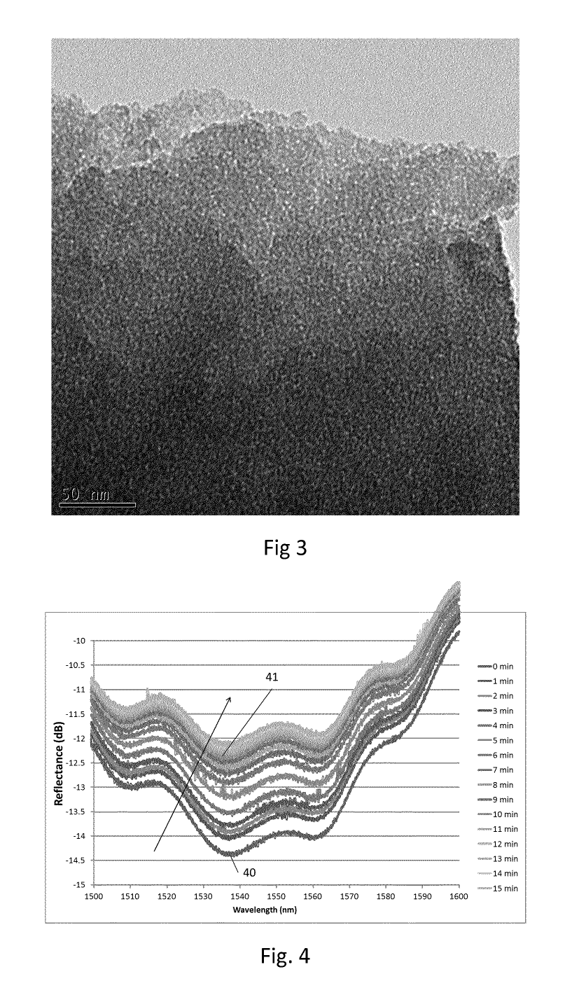

FIG. 3 shows an image of a porous matrix. As can be seen from the scale indicating 50 nm, the porous matrix has pores having an average diameter of between 4 and 6 nm.

FIG. 4 shows reflectance (in dB) as a function of wavelength for the gas sensor of example 1. Each curve shows the reflectance measured after a different time delay after the sensor is exposed to the mixture of 3 ppm of NO₂ in test air. The curve (40) is the curve of reflectance at 0 minute i.e. stable conditions when held in test air with no NO₂, the curve (41) is the curve of the reflectance after 10 minutes of continuous exposure to the gas flow consisting of a mixture of 3 ppm of NO₂ in test air, while the intervening curves are reflectance at successive one minute intervals between 0 and 10 minutes. The reflectance is shown in a preferred range of detection wavelengths, between 1500 nm and 1600 nm. For example, the change of reflectance between the curve (40) and the curve (41) at a detection wavelength 1536 nm is about 2 dB.

FIG. 5 shows the evolution in time of the reflectance (in dB) of the gas sensor subjected to cycles of

i) being exposed to the mixture of 3 ppm of NO₂ in test air for a short time (about 15 minutes) e.g. starting at the position indicated at 50

ii) subsequently being held in a stream of test air with no NO₂ present e.g. starting at the position indicated at 50′

at the detection wavelength 1540 nm.

Just prior to the start of the second cycle illustrated, the reflectance indicated at 51 has reverted to about 90% of the initial reflectance at 50 after about 85 minutes in test air. The second cycle then begins, the sensor being exposed again to the gas stream comprising a mixture of 3 ppm of NO₂ in test air for about 15 minutes during which time the reflectance again rises before the gas stream is switched back to test air with no NO₂ present causing the reflectance to fall back to approximately the value indicated at 51 after about 85 minutes.

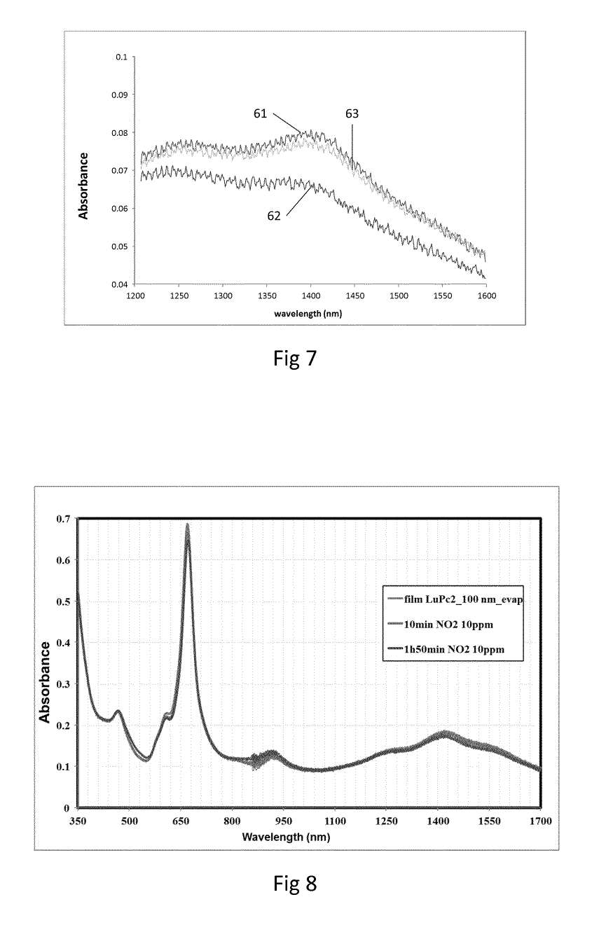

FIG. 6 and FIG. 7 show the wavelength of the absorbance of the gas sensor at three different statuses:
curve 61: exposed to test air (with no NO₂ present)
curve 62: 2 minutes after continuous exposure to a mixture of 10 ppm of NO₂ in test air
curve 63: 8 hours after subsequent exposure to test air (with no NO₂ present).

The change in absorbance in the wavelengths 1200-1600 nm shown by curve 62 allows monitoring at these wavelengths.

FIG. 8 shows the wavelength of the absorbance of a solid layer of LuPc₂ (ie not held within a porous matrix) at three different statuses: in test air, 10 minutes after the continuous exposure to 10 ppm of NO₂, 110 minutes after the continuous exposure to NO₂. The optical change is very small and thus difficult to detect.

1-18. (canceled)

19. A gas sensor comprising an optical fibre and a gas sensitive detection material at a portion of the surface of the optical fibre, said gas sensitive detection material comprising a gas sensitive reactant in a porous matrix, wherein the gas sensitive detection material undergoes a reversible change of reflectance and/or absorbance at a detection wavelength when exposed to a gas to be detected in a gaseous atmosphere.

20. The gas sensor of claim 19, wherein the gaseous atmosphere is ambient air.

21. The gas sensor of claim 19 wherein the gas sensitive reactant comprises a lanthanide bisphtalocyanine.

22. The gas sensor of claim 21, wherein the gas sensitive reactant comprises lutetium bisphthalocyanine (LuPc₂).

23. The gas sensor of claim 19, wherein the gas to be detected is selected from the group consisting of: a nitrogen oxide, NO₂, O₃, CO, formaldehyde, NH₃ and mixtures thereof.

24. The gas sensor of claim 19, wherein the gas sensor is configured to detect a gas in a gaseous atmosphere at a temperature from −30° C. to 45° C.

25. The gas sensor of claim 19, wherein the gas to be detected comprises an oxidising gas.

26. The gas sensor of claim 25, wherein the gas to be detected comprises an oxidising gas selected from the group consisting of: a nitrogen oxide, NO₂, O₃ and mixtures thereof.

27. The gas sensor of claim 19, wherein the gas to be detected comprises a reducing gas.

28. The gas sensor of claim 27, wherein the gas to be detected comprises a reducing gas selected from the group consisting of: CO, formaldehyde, NH₃ and mixtures thereof.

29. The gas sensor of claim 19, wherein the pores of the porous matrix have an average diameter in the range 4-100 nm.

30. The gas sensor of claim 19, wherein the gas sensitive detection material has a porosity in the range 20-60%.

31. The gas sensor of claim 19, wherein the change of reflectance and/or absorbance of the gas sensitive detection material at the detection wavelength is ≧10% in less than 10 minutes when the gas sensitive detection material is exposed to at least 10 ppm of the gas to be detected.

32. The gas sensor of claim 19, wherein the change of reflectance and/or absorbance of the gas sensitive detection material at the detection wavelength is ≧10% in less than 5 minutes when the gas sensitive detection material is exposed to at least 10 ppm of the gas to be detected.

33. The gas sensor of claim 19, wherein the change of reflectance and/or absorbance of the gas sensitive detection material at the detection wavelength is ≧10% in less than 2 minutes when the gas sensitive detection material is exposed to at least 10 ppm of the gas to be detected.

34. The gas sensor of claim 19, wherein the difference between the refractive index of the gas sensitive detection material and the refractive index of the optical fibre at the interface between the gas sensitive detection material and the optical fibre, at the detection wavelength, is less than that 10%.

35. The gas sensor of claim 19, wherein the detection wavelength is in the range 300 nm to 1700 nm.

36. The gas sensor of claim 19, wherein the detection wavelength is in the range 1100 nm to 1600 nm.

37. The gas sensor of claim 19, wherein the detection wavelength is in the range 1380 nm to 1550 nm.

38. The gas sensor of claim 19, wherein the thickness of the gas sensitive detection material is in the range 50 nm-15 μm.

39. The gas sensor of claim 19, wherein the optical fibre comprises a plurality of spaced detection zones, each detection zone comprising a gas sensitive detection material at a portion of the surface of the optical fibre and an associated grating.

40. The gas sensor of claim 19, wherein the optical fibre comprises a plurality of spaced detection zones, each detection zone comprising a gas sensitive detection material at a portion of the surface of the optical fibre and an associated grating selected from the group consisting of: a Fibre Bragg Grating (FBG), a Long Period Fibre Grating (LPFG) and Tilted Fibre Bragg Grating (TFBG).

41. The gas sensor of claim 19 wherein the gas sensor further comprises a gas filter between the gas sensitive detection material and the gaseous atmosphere, the filter being adapted to reduce the concentration of the gas to be in detected in contact with the gas sensitive detection material with respect to the concentration of the gas to be in detected present in the gaseous atmosphere.

42. The gas sensor of claim 41 wherein the gas filter comprises activated carbon.

43. The gas sensor of claim 19, wherein the optical fibre is a silica optical fibre.

44. A method of detecting a gas in a gaseous atmosphere comprising:
arranging the gas sensor of claim 19 in the gaseous atmosphere;
Transmitting an incident spectrum including at least one detection wavelength through the optical fibre;
Collecting at least a portion of the reflectance spectrum and/or transmittance spectrum;
Comparing the at least a portion of the reflectance and/or reflectance spectrum with the incident spectrum at the at least one detection wavelength.

45. The method of detecting a gas of claim 44 comprising subjecting the gas sensitive detection material to UV radiation subsequent to its exposure to the gas to be detected.
